(19) United States
(12) Patent Application Publication
(10) Pub. No.: US 2023/0256171 A1
(43) Pub. Date: Aug. 17, 2023

(54) NEEDLE-FREE INJECTOR

(71) Applicant: JEISYS MEDICAL INC., Seoul (KR)

(72) Inventors: Myeong Wook GU, Seoul (KR); Yeong Gi JEON, Seoul (KR); Kyu Young SEO, Seoul (KR); Seung Wook LEE, Seoul (KR); Si Youn KIM, Seoul (KR); Won Ju YI, Seoul (KR); Dong Hwan KANG, Seoul (KR)

(73) Assignee: JEISYS MEDICAL INC., Seoul (KR)

(21) Appl. No.: 18/302,160

(22) Filed: Apr. 18, 2023

Related U.S. Application Data
(63) Continuation of application No. PCT/KR2021/014702, filed on Oct. 20, 2021.

(30) Foreign Application Priority Data
Nov. 6, 2020 (KR) 10-2020-0147454

(51) Int. Cl. A61M 5/30; A61M 5/19; A61M 5/315

(52) U.S. Cl. CPC A61M 5/30; A61M 5/19; A61M 5/31576

(57) ABSTRACT

Disclosed is a needle-free injector including a power supply unit, a coil that receives current from the power supply unit, a drug chamber in which a drug is positioned, a housing connected to the drug chamber, a separation membrane that separates the housing and the drug chamber, a metal material adjacent to a separation membrane, and an injection unit positioned in the drug chamber to inject the drug. The coil is positioned outside the housing or outside the drug chamber.

### CROSS-REFERENCE TO RELATED APPLICATIONS

The present application is a continuation of International Patent Application No. PCT/KR2021/014702, filed on Oct. 20, 2021, which is based upon and claims the benefit of priority to Korean Patent Application No. 10-2020-0147454 filed on Nov. 6, 2020. The disclosures of the above-listed applications are hereby incorporated by reference herein in their entirety.

### BACKGROUND

Embodiments of the inventive concept described herein relate to a needle-free injector.

A drug delivery system is designed to efficiently deliver the required amount of drug into a body such that side effects occurring in an existing method are minimized and therapeutic effects of medicines are maximized, when medicines for treating diseases or wounds in a human body are used.

An injection method most commonly used in the drug delivery system may inject drugs accurately and efficiently. However, the injection method has several disadvantages, such as injection phobia due to pain during injection, risk of infection due to reuse, and a large amount of medical waste.

To solve these issues, drug delivery methods such as needle-free injectors are being developed.

For example, a liquid injection technology that is one of the needle-free injector technologies is a technology that thermally expands liquid by applying shock waves through laser or electric waves to the liquid and generates a high-speed liquid stream by using the pressure generated during the thermal expansion so as to inject the liquid into a skin.

However, the conventional liquid injection technology has a complicated structure and is difficult to be miniaturized.

### SUMMARY

The present disclosure has been made to solve the above-mentioned problems occurring in the prior art, an object of the present disclosure is to present disclosure provide a needle-free injector which has a simple structure and is easy to be miniaturized by using a metal material.

Another object of the present disclosure is to present disclosure provide a needle-free injector that may inject a drug through attractive force or repulsive force between a metal material and a solenoid electromagnet formed in a coil when a current is applied, and may restore into an original form by the elasticity of a separation membrane.

According to an embodiment, a needle-free injector includes a power supply unit, a coil that receives a current from the power supply unit, a drug chamber in which a drug is positioned, a housing connected to the drug chamber, a separation membrane that separates the housing and the drug chamber, a metal material adjacent to a separation membrane, and an injection unit positioned in the drug chamber to inject the drug. The coil is positioned outside the housing or outside the drug chamber.

According to an embodiment, a needle-free injector includes a power supply unit that applies current, a coil that forms an electromagnet by receiving the current from the power supply unit, a separation membrane adjacent to the coil, and a permanent magnet provided between the separation membrane and the coil and positioned on the separation membrane. When the current is applied by the power supply unit, repulsive force acts between the coil forming the electromagnet and the permanent magnet such that the drug is injected.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1A is a cross-sectional view schematically illustrating that current is not applied to a needle-free injector, according to an embodiment of the present disclosure;

FIG. 1B is a cross-sectional view schematically illustrating that a drug is injected by applying current to a needle-free injector, according to an embodiment of the present disclosure;

FIG. 2A is a cross-sectional view schematically illustrating that current is not applied to a needle-free injector, according to an embodiment of the present disclosure; and

FIG. 2B is a cross-sectional view schematically illustrating that a drug is injected by applying current to a needle-free injector, according to an embodiment of the present disclosure.

### DETAILED DESCRIPTION

The above and other aspects, features and advantages of the present disclosure will become apparent from embodiments to be described in detail in conjunction with the accompanying drawings. The present disclosure, however, may be embodied in various different forms, and should not be construed as being limited only to the illustrated embodiments. Rather, these embodiments are provided as examples so that the present disclosure will be thorough and complete, and will fully convey the scope of the present disclosure to those skilled in the art. The present disclosure may be defined by the scope of the claims.

The terms used herein are provided to describe embodiments, not intended to limit the present disclosure. In the specification, the singular forms include plural forms unless particularly mentioned. The terms “comprises” and/or “comprising” used herein do not exclude the presence or addition of one or more other components, in addition to the aforementioned components. The same reference numerals denote the same components throughout the specification. As used herein, the term “and/or” includes each of the associated components and all combinations of one or more of the associated components. It will be understood that, although the terms “first”, “second”, etc., may be used herein to describe various components, these components should not be limited by these terms. These terms are only used to distinguish one component from another component. Thus, a first component that is discussed below could be termed a second component without departing from the technical idea of the present disclosure.

Unless otherwise defined, all terms (including technical and scientific terms) used herein have the same meaning as commonly understood by those skilled in the art to which the present disclosure pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, embodiments of the present disclosure will be described in detail with reference to accompanying drawings.

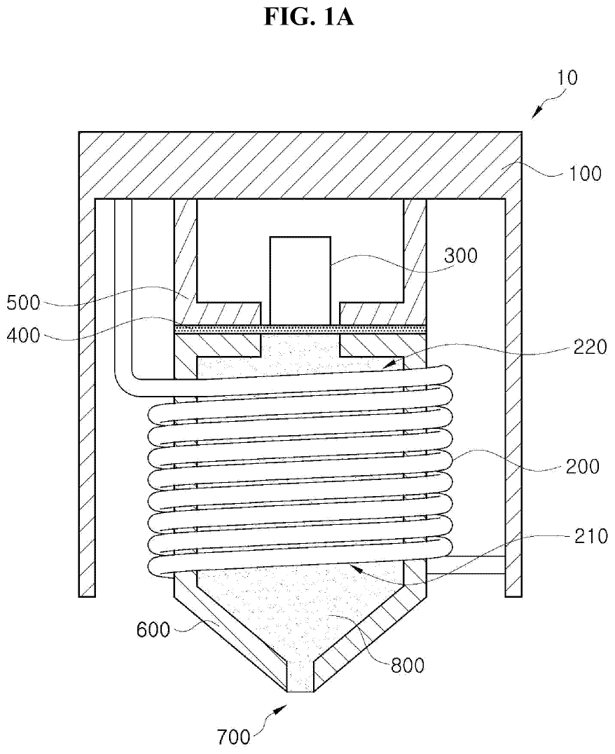

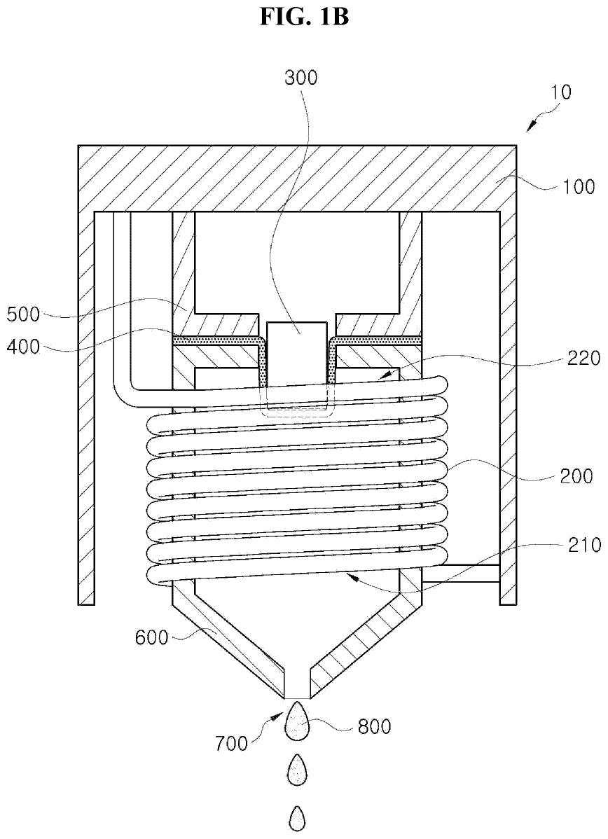

FIG. 1A is a cross-sectional view schematically illustrating that a current is not applied to a needle-free injector, according to an embodiment of the present disclosure. FIG. 1B is a cross-sectional view schematically illustrating that a drug is injected by applying current to a needle-free injector, according to an embodiment of the present disclosure.

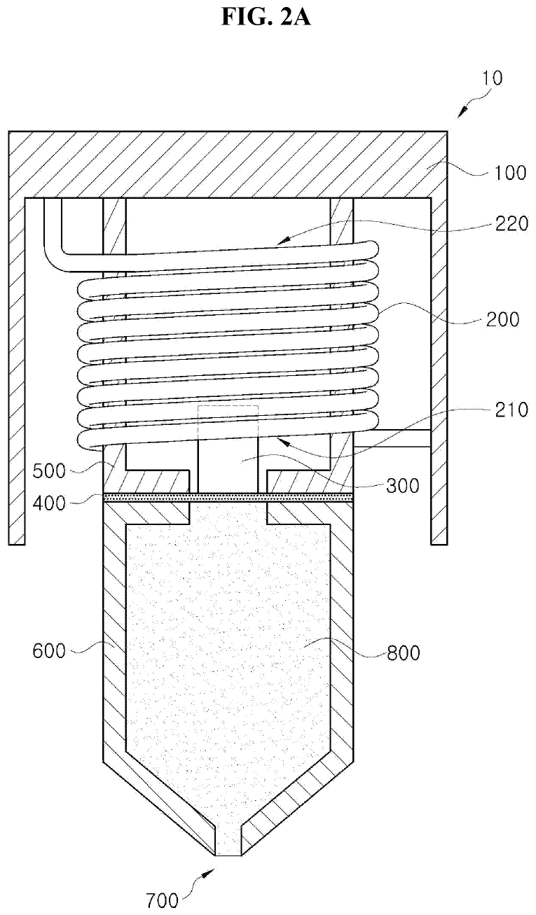

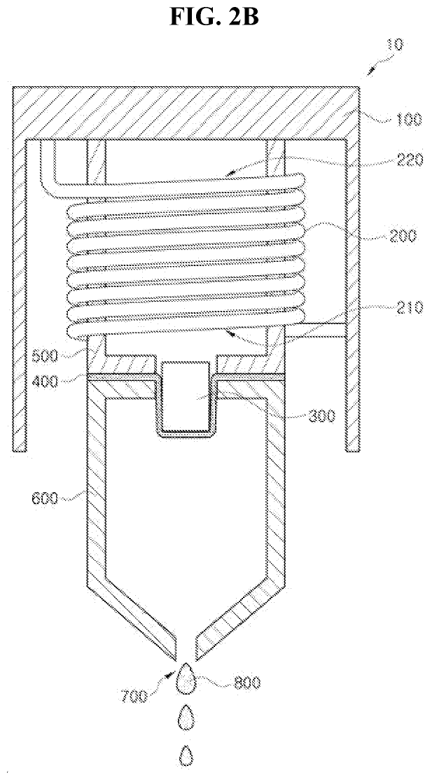

FIG. 2A is a cross-sectional view schematically illustrating that a current is not applied to a needle-free injector, according to an embodiment of the present disclosure. FIG. 2B is a cross-sectional view schematically illustrating that a drug is injected by applying current to a needle-free injector, according to an embodiment of the present disclosure.

Referring to FIGS. 1A, 1B, 2A, and 2B, a needle-free injector 10 according to an embodiment of the present disclosure includes a power supply unit 100, a coil 200, a metal material 300, and a separation membrane 400. The needle-free injector 10 according to an embodiment of the present disclosure includes a housing 500, a drug chamber 600, and an injection unit 700.

The power supply unit 100 applies a current. The power supply unit 100 may include a positive electrode and a negative electrode. For example, the power supply unit 100 may be a battery.

For example, the current may be a pulse current. The “pulse current” may be “pulsed power”, which may increase instantaneous power by emitting a large amount of energy in a short time after energy is accumulated. At this time, the width of the emitted pulse may be in units of several milliseconds to several nanoseconds. Preferably, the width of the emitted pulse is a unit of several microseconds to several nanoseconds.

Although not shown in drawings, the power supply unit 100 may include a power supply unit that supplies voltage and current, an electricity storage unit that stores electricity supplied from the power supply unit, and a switch that applies electrical energy, which is stored in the electricity storage unit, to the pulsed power. The power supply unit 100 may further include an electric circuit that maintains the form of the generated pulse, and the electrical circuit may be a pulse forming network (PFN).

The electricity storage unit may be preferably one or more selected from a capacitor and an inductor. The PFN may maintain the form of a pulse by preventing the form of a square pulse from collapsing due to parasitic inductance.

Although not shown, when the switch is turned on, current may be applied from the power supply unit 100 to the coil 200. When the switch is turned off, the current applied from the power supply unit 100 to the coil 200 may be cut off.

The coil 200 receives the current from the power supply unit 100 to form an electromagnet.

The coil 200 may be wound with a linear material having good electrical conductivity.

When the current is applied by the power supply unit 100, a magnetic field is formed around the coil 200.

For example, a left portion of the coil 200 may be connected to the positive electrode of the power supply unit 100, and a right portion thereof may be connected to the negative electrode of the power supply unit 100.

The coil 200 is positioned outside the housing 500 or outside the drug chamber 600.

Referring to FIGS. 1A and 1B, the coil 200 is positioned outside the drug chamber 600. The coil 200 is positioned under the separation membrane 400.

Referring to FIGS. 2A and 2B, the coil 200 is positioned outside the housing 500. The coil 200 is positioned on the separation membrane 400.

Returning to FIGS. 1A, 1B, 2A, and 2B, the metal material 300 is provided between the separation membrane 400 and the coil 200. The metal material 300 is positioned on the separation membrane 400. The metal material 300 may be one selected from a permanent magnet and a conductor.

When current is applied to the coil 200, a solenoid electromagnet is formed, and thus attractive force or repulsive force may occur between the coil 200 and the metal material 300. Accordingly, the separation membrane 400 and the metal material 300 may move from the housing 400 to the drug chamber 600, and thus a drug 800 may be injected through the injection unit 700.

When current is applied to the coil 200 in the case in which the metal material 300 is a permanent magnet, a solenoid electromagnet may be formed, and thus repulsive force may be generated between the coil 200 and the permanent magnet by Lorentz force. Accordingly, the separation membrane 400 and the permanent magnet may move from the housing 400 to the drug chamber 600, and thus the drug 800 may be injected through the injection unit 700.

The separation membrane 400 is positioned close to the coil 200. The separation membrane 400 is provided between the housing 500 and the drug chamber 600. For example, in a case in which the housing 500 is integrated with the drug chamber 600, the separation membrane 400 may separate the housing 500 and the drug chamber 600.

The separation membrane 400 is not altered or damaged by the repulsive force between the coil 200 and the metal material 300. The separation membrane 400 may move by the repulsive force between the coil 200 and the metal material 300.

The separation membrane 400 has elasticity. The separation membrane 400 may move from the housing 500 to the drug chamber 600 by the repulsive force between the coil 200 and the metal material 300 and may apply pressure to the inside of the drug chamber 600.

For example, the separation membrane 400 may be made of natural rubber or synthetic rubber harmless to a human body.

The housing 500 has an enclosed accommodation space. When power is not supplied from the power supply unit 100, the metal material 300 may be positioned inside the housing 500.

For example, the housing 500 may be approximately cylindrical. A lower portion of the housing 500 may be connected to the drug chamber 600. The separation membrane 400 may be positioned at the bottom of the housing 500.

The drug chamber 600 has an enclosed accommodation space. The drug 800 is contained in the drug chamber 600. For example, the drug chamber 600 may be approximately cylindrical. The separation membrane 400 may be disposed on the drug chamber 600. The lower portion of the drug chamber 600 may be connected to the injection unit 700. One side of the drug chamber 600 may be connected to a drug supply unit.

For example, the drug 800 contained in the drug chamber 600 may correspond to an amount to be injected once. When current is applied and then the entire drug 800 contained in the drug chamber 600 is injected through the injection unit 700, the drug supply unit may resupply the amount to be injected once to the drug chamber 600. However, it is not limited thereto. For example, the drug 800 contained in the drug chamber 600 may correspond to an amount to be injected twice or more.

When the pressure inside the housing 500 increases, the pressure is applied into the drug chamber 600. In other words, the pressure inside the drug chamber 600 may increase. Accordingly, the pressure may be applied to the drug 800. Accordingly, the drug 800 may be injected through the injection unit 700 into a user. This will be described later in more detail.

The injection unit 700 is positioned in the drug chamber 600. For example, the injection unit 700 may be defined as a hole at the bottom of the drug chamber 600. However, it is not limited thereto. For example, in a case in which the injection unit 700 is capable of injecting a drug, the injection unit 700 may be connected to the drug chamber 600 and may protrude from the top of the drug chamber 600 to the bottom thereof. The injection unit 700 injects the drug 800. The injection unit 700 may inject the drug 800 from the housing 500 into the drug chamber 600.

The diameter of the injection unit 700 may be within the range of 50 micrometers to 1000 micrometers. In a case in which the diameter of the injection unit 700 is less than 50 micrometers, the amount of the drug 800 injected may be small and the drug 800 may not be injected to a sufficient depth in a body of the user receiving the drug 800. In a case in which the diameter of the injection unit 700 is greater than 1000 micrometers, the diameter of the injected microjet increases, and thus the amount of the drug 800 that bounces off on the surface of a skin increases, and thus a lot of the drug 800 may be wasted.

When current is applied to the power supply unit 100, repulsive force acts between the coil 200 forming an electromagnet and the metal material 300, and thus the needle-free injector 10 according to an embodiment of the present disclosure injects the drug 800. This will be described later in more detail.

Although not shown, the needle-free injector 10 according to an embodiment of the present disclosure may further include a drug supply unit. The drug supply unit receives the drug 800 from a drug storage unit and provides the drug 800 to the drug chamber 600. For example, the drug supply unit may be connected to a side surface of the drug chamber 600.

Although not shown in drawings, the needle-free injector 10 according to an embodiment of the present disclosure may further include a drug storage unit and a check valve.

The drug storage unit may store the drug 800 provided in the drug chamber 600. The drug storage unit may be connected to the drug supply unit.

The check valve may allow the drug 800 to be delivered from the drug supply unit to only the drug chamber 600. For example, the check valve prevents the drug 800 from being delivered from the drug chamber 600 to the drug supply unit. For example, the check valve may be positioned inside the drug supply unit.

Hereinafter, a method of injecting a drug in a needle-free injector according to an embodiment of the present disclosure will be described in more detail.

Referring to FIGS. 1A and 1B, as described above, the coil 200 may be positioned outside the drug chamber 500. When the current is applied by the power supply unit 100, a magnetic field is formed around the coil 200. The coil 200 receives the current from the power supply unit 100 to form a solenoid electromagnet.

When the solenoid electromagnet is formed, attractive force is generated between one end 220 of the coil 200 adjacent to the separation membrane 400 and the metal material 300. Accordingly, the metal material 300 and the separation membrane 400 adjacent to the metal material 300 move from the housing 500 to the drug chamber 600. The drug 800 contained in the drug chamber 600 is injected through the injection unit 700 under pressure.

When the current is cut off by the power supply unit 100, the metal material 300 and the separation membrane 400 are restored to their original positions, due to the elasticity of the separation membrane 400. When the metal material 300 and the separation membrane 400 are restored to their original positions, the drug 800 may be supplied from the drug supply unit to the drug chamber 600.

Referring to FIGS. 2A and 2B, as described above, the coil 200 may be positioned outside the housing 500. When the current is applied by the power supply unit 100, a magnetic field is formed around the coil 200. The coil 200 receives the current from the power supply unit 100 to form a solenoid electromagnet.

When the solenoid electromagnet is formed, repulsive force is generated between one end 210 of the coil 200 adjacent to the separation membrane 400 and the metal material 300. Accordingly, the metal material 300 and the separation membrane 400 adjacent to the metal material 300 move from the housing 500 to the drug chamber 600. The drug 800 contained in the drug chamber 600 is injected through the injection unit 700 under pressure.

When the current is cut off by the power supply unit 100, the metal material 300 and the separation membrane 400 are restored to their original positions due to the elasticity of the separation membrane 400. When the metal material 300 and the separation membrane 400 are restored to their original positions, the drug 800 may be supplied from the drug supply unit to the drug chamber 600.

The needle-free injector 10 according to an embodiment of the present disclosure may have a simple structure and be easy to be miniaturized since using a metal material.

The needle-free injector 10 according to an embodiment of the present disclosure does not require a large device structure and expensive equipment costs since using a metal material.

When applying current is by pulsed power that is a high voltage, the needle-free injector 10 according to an embodiment of the present disclosure may instantaneously form a solenoid electromagnet in a coil and may easily inject a drug through attraction force or repulsive force between the solenoid electromagnet and the metal material. Besides, the needle-free injector 10 according to an embodiment may be restored to its original form without additional equipment or external force due to the elasticity of the separation membrane.

Although an embodiment of the present disclosure are described with reference to the accompanying drawings, it will be understood by those skilled in the art to which the present disclosure pertains that the present disclosure may be carried out in other detailed forms without changing the scope and spirit or the essential features of the present disclosure. Therefore, the embodiments described above are provided by way of example in all aspects, and should be construed not to be restrictive.

According to an embodiment of the present disclosure, a needle-free injector which has a simple structure and is easy to be miniaturized may be provided by using a metal material.

According to an embodiment of the present disclosure, a needle-free injector may inject a drug through attractive force or repulsive force between a metal material and a solenoid electromagnet formed in a coil when a current is applied, and may restore into an original form by the elasticity of a separation membrane.

What is claimed is:

1. A needle-free injector comprising:
a power supply unit;
a coil configured to receive current from the power supply unit;
a drug chamber in which a drug is positioned;
a housing connected to the drug chamber;
a separation membrane configured to separate the housing and the drug chamber;
a metal material adjacent to the separation membrane; and
an injection unit positioned in the drug chamber and configured to inject the drug,
wherein the coil is positioned outside the housing or outside the drug chamber.

2. The needle-free injector of claim 1, wherein, when current is applied to the coil by the power supply unit, magnetism is formed by the coil, and
wherein the metal material and the separation membrane move from the housing to the drug chamber by the magnetism, and the drug is injected through the injection unit.

3. The needle-free injector of claim 2, wherein, when the current applied by the power supply unit is cut off, the metal material and the separation membrane are restored to their original positions.

4. The needle-free injector of claim 1, wherein the power supply unit applies a pulse current.

5. The needle-free injector of claim 4, wherein the pulse current is pulsed power.

6. The needle-free injector of claim 1, wherein, when current is applied by the power supply unit, the coil forms a solenoid electromagnet.

7. The needle-free injector of claim 1, wherein the metal material is one selected from a permanent magnet and a conductor.

8. The needle-free injector of claim 1, further comprising:
a drug supply unit connected to the drug chamber and configured to supply the drug to the drug chamber.

9. The needle-free injector of claim 1, wherein the coil is positioned under the separation membrane and is positioned outside the drug chamber.

10. The needle-free injector of claim 9, wherein the metal material is one selected from a permanent magnet and a conductor.

11. The needle-free injector of claim 9, wherein, in a case in which one surface of the metal material is coupled to the separation membrane, when the current is applied by the power supply unit, different poles are respectively formed at one end of the coil relatively close to the separation membrane and the other end of the coil relatively far from the separation membrane, and the permanent magnet moves from the housing to the drug chamber through attractive force acts between the electrode formed at the one end of the coil and an electrode of the metal material such that the drug is injected through the injection unit.

12. The needle-free injector of claim 1, wherein the coil is positioned on the separation membrane and is positioned outside the housing.

13. The needle-free injector of claim 12, wherein the metal material is a permanent magnet.

14. The needle-free injector of claim 12, wherein, in a case in which one surface of the metal material is coupled to the separation membrane, when the current is applied by the power supply unit, different poles are respectively formed at one end of the coil relatively close to the separation membrane and the other end of the coil relatively far from the separation membrane, and the permanent magnet moves from the housing to the drug chamber through repulsive force acts between the electrode formed at the one end of the coil and an electrode of the metal material such that the drug is injected through the injection unit.

15. The needle-free injector of claim 1, wherein the drug contained in the drug chamber corresponds to an amount to be injected once.

16. The needle-free injector of claim 1, wherein, when the current is cut off by the power supply unit, the separation membrane is restored to its original position.

17. A needle-free injector comprising:
a power supply unit configured to apply current;
a coil configured to form an electromagnet by receiving the current from the power supply unit;
a separation membrane adjacent to the coil; and
a permanent magnet provided between the separation membrane and the coil and positioned on the separation membrane,
wherein, when the current is applied by the power supply unit, repulsive force acts between the coil forming the electromagnet and the permanent magnet such that the drug is injected.
